(19) United States
(12) Patent Application Publication
(10) Pub. No.: US 2013/0149359 A1
(43) Pub. Date: Jun. 13, 2013

(54) Effervescent Mouthwash

(71) Applicant: Eric M. Sanders, Canton, GA (US)

(72) Inventor: Eric M. Sanders, Canton, GA (US)

(21) Appl. No.: 13/316,600

(22) Filed: Dec. 12, 2011

(51) Int. Cl. A61K 9/00; A61K 9/48; A61Q 11/00; A61K 8/02

(52) U.S. Cl. 424/401; 424/400; 424/451; 424/44

(57) ABSTRACT

The present invention comprises an effervescent composition containing an antiseptically effective amount of phytochemicals, flavonoids, and/or antimicrobial agents comprising an effective amount of polyphenols derived from a plant selected from the group consisting of White tea, Green tea, Oolong tea, Black tea, camellia sinensis, thea sinensis, thea bohea, thea assamica, camellia thea, and combinations thereof. An effervescent powder, tablet, capsule film. Strip, or coating of effervescent powder for oral use is produced containing a pharmaceutically acceptable carbon dioxide source selected from the group consisting of a bicarbonate salt, a carbonate salt, and mixtures thereof; an organic acid and flavonoids and polyphenols derived from a plant selected from the group consisting of White tea, Green tea, Oolong tea, Black tea, camellia sinensis, thea sinensis, thea bohea, thea assamica, camellia thea, and combinations thereof. Desired profiles of catechins and the products of fermentation may be produced. The products of the present invention may be used by humans and/or their pets and other animals.

### CROSS REFERENCE TO RELATED APPLICATIONS

None

### FIELD OF THE INVENTION

The present invention relates to the field of oral hygiene and health care preparations for people, pets and animals; and more particularly to new and improved oral health care preparations comprising effervescent compositions and mouthwashes.

### BACKGROUND OF THE INVENTION

Although considerable effort has been made to improve the various cleaning and aesthetic characteristics of oral hygiene compositions, one area for improving both the cleaning action and the aesthetic nature or oral hygiene compositions has found little development. This area relates to the development of effervescent compositions.

U.S. Re. Pat. No. 14,961, reissued Oct. 19, 1920 on U.S. Pat. No. 1,297,494, discloses a dentifrice containing a solid acidic substance and a carbonate. The chemical action of the compounds provides an effervescence.

U.S. Pat. No. 3,629,468 discloses an effervescent mouthwash tablet. The mouthwash tablet contains an acid compound and a salt capable of evolving carbon dioxide when brought into aqueous reactive contact with the acid component.

Howell, U.S. Pat. No. 3,962,417 teaches an effervescent dentifrice for treatment of and fighting against caries.

The U.S. Pat. No. 4,267,164 teaches an effervescent oral composition for the convenient and effective delivery of fluoride ions to the oral cavity.

Welsh, U.S. Pat. No. 3,518,343 teaches an effervescent mouthwash tablet composition in tablet form, where the binder is a specific combination of ingredients.

The prior art above teaches that combinations of baking soda and organic acids (typically fruit acids) begin to immediately effervesce in water and when placed in the oral cavity thereby chemically and/or mechanically remove plaque and food debris during effervescence in the mouth.

### BRIEF SUMMARY OF THE INVENTION

The present invention comprises an effervescent composition containing an antiseptically effective amount of phytochemicals, flavonoids, polyphenols, and/or antimicrobial agents comprising as a preferred embodiment the polyphenols derived from a plant selected from the group consisting of white tea, green tea, oolong tea, black tea, camellia sinensis, thea sinensis, thea bohea, thea assamica, camellia thea, and combinations thereof.

The present invention may be presented as an effervescent powder, tablet or capsule of or containing as effervescent powder for oral use, comprising a pharmaceutically acceptable carbon dioxide source selected from the group consisting of a bicarbonate salt, a carbonate salt and mixtures thereof; a food grade or organic acid and an antiseptically effective amount of polyphenols derived from a plant selected from the group consisting of white tea, green tea, oolong tea, black tea, camellia sinensis, thea sinensis, thea bohea, thea assamica, camellia thea, and combinations thereof, together with other compatible agents, flavors, and additives for various specific purposes.

The present invention may be used by or on persons (human beings), pet and/or animals.

### Objects

Pursuant to the foregoing, it may be regarded as an object of the present invention to overcome the deficiencies of and provide for improvements in the state of the prior art as described above and as may be known to those skilled in the art.

It is a further object of the present invention to utilize a process and apparatus in accordance with the present objects which may be readily carried out with comparatively simple equipment and with relatively simple engineering requirements.

Still further objects may be recognized and become apparent upon consideration of the following specification, taken as a whole, in conjunction with the claims, wherein by way of illustration and example, an embodiment of the present invention is disclosed.

It is an object of the present invention to provide an oral wash composition that may be inserted into the oral cavity after brushing and/or following a meal, snack, coffee or smoking in order to rinse the mouth free of debris and inhibit the formation of caries and plaque.

It is a further object of the present invention to provide a good tasting efficacious oral composition that is safe for human and animal consumption and that will be used regularly by individuals or animals in preventing disease and or providing good hygiene.

It is a further object of the present invention to provide an effervescent oral composition that is effective in delivering the benefits of a plant containing an effective amount of polyphenols derived from a plant selected from the group consisting of white tea, green tea, oolong tea, black tea, camellia sinensis, thea sinensis, thea bohea, thea assamica, camellia thea, and combinations thereof.

It is a further object of the present invention to provide an effervescent oral composition that is effective in delivering the benefits of a plant containing an effective amount of polyphenols derived from a plant selected from the group consisting of white tea, green tea, oolong tea, black tea, camellia sinensis, thea sinensis, thea bohea, thea assamica, camellia thea, and combinations thereof, together with other compatible agents, flavors, and additives for various specific purposes to humans and/or animals.

The above and other objects of the present invention are realized and the limitations of the prior art are overcome by providing a new and improved effervescent composition containing polyphenols derived from a plant consisting of white tea, green tea, oolong tea, black tea, camellia sinensis, thea sinensis, thea bohea, thea assamica, camellia thea, and combinations thereof, together with other compatible agents, flavors, and additives for various specific purposes.

### GENERAL DESCRIPTION OF THE PRESENT INVENTION

For a further understanding of the nature, function, and objects of the present invention, reference will now be made to the following detailed description taken in conjunction with the accompanying claims. A detailed description of a preferred embodiment is provided herein, as well as, the best mode of carrying out and employing the present invention. It is to be understood, however, that the present invention may be embodied in various forms and by various ways and means. Therefore, specific details disclosed herein are not to be interpreted as limiting, but rather as a basis for the claims and as a representative basis for teaching one skilled in the art to employ the present invention in virtually any appropriately detailed system, structure or manner. The practice of the present invention is illustrated by the following examples which are deemed illustrative of both the process taught by the present invention and the product or article of manufacture yielded in accordance with the present invention.

While the specification concludes with claims, as required, it is believed that the present invention can be best understood by reference to the following description.

All documents cited are, in relevant part, incorporated herein by reference. The citation of any document is not to be construed as an admission that it is, or is a part of the, prior art with respect to the present invention.

As used herein, the term “comprising” means that other steps and other ingredients which do not affect the end result can be added or conjointly employed in the compositions of the present invention as long as the materials perform their intended functions. This term encompasses the terms “consisting of” and “consisting essentially of” in its and their intended use.

As used herein, “and”, unless otherwise noted or dictated by context, shall be the “several and” which is to say that “A and B” means A and B, jointly or severally.

Further as used herein “or” unless otherwise noted or dictated by context, shall be the “inclusive or” which is to say that “A or B” means A or B, or both and shall further include the “equivalent or” where B is equivalent to A in some or all respects.

Unless otherwise noted, all percentages and ratios used in the specification and claims are by weight of the total composition and all measurements are made at 25 degrees C.

The term “safe and effective amount” as used herein means an amount of a compound, component, or composition sufficient to significantly induce a positive benefit, but low enough to avoid serious side effects, i.e., to provide a reasonable risk to benefit ratio, within the scope of sound medical judgment, while being generally safe to the hard and soft tissues of the oral cavity.

The term “orally active” as used herein means a material that provides either a cosmetic, prophylactic or therapeutic benefit within the oral cavity.

The term “acceptable carrier”, as used herein, means a suitable material which can be used to apply or transport the agents of the present invention to or within the oral cavity without undue toxicity, irritation, allergic response or the like commensurate with a reasonable risk/benefit ratio.

The present invention consists of an effervescent composition containing polyphenols derived from a plant selected from the group consisting of white tea, green tea, oolong tea, black tea, camellia sinensis, thea sinensis, thea bohea, thea assamica, camellia thea, and combinations thereof, together with other compatible agents, flavors, and additives for various specific purposes.

The composition may be in powdered form or in the form of a tablet, film or gel coated tablet, capsule, or coated micro-spheres.

As used herein, “film” refers to a thin, membranous skin which is thin in comparison with its other dimensions, having a nominal maximum thickness of 250 micrometers to a lower limit of a thickness on the order of about 25 micrometers. It may be any thin layer covering, coating or wrapping which may enter or reside on the structure or reside on, in or near the surface of a substrate structure, usually as a monomolecular layer.

As used herein , a “gel” refers to a two phase colloidal system consisting of a solid and a liquid I a more solid form than a sol—semi-solid in nature, rich in liquid. A “gel coat” is a resin or resin like coating applied to the surface of a substrate to become a part of the finished laminate.

Films and gel coats may be used to coat a particulate mix, tablet, capsule, microsphere or the like. They may form films or strips for use as breath fresheners and the like. Such films or strips may be used in and around dog and cat toys and the like. They may be used on the surface and/or as a part of a laminate structure comprising the toy or amusement device.

The present invention provides the benefits associated with polyphenols derived from plant selected from the group consisting of white tea, green tea, oolong tea, black tea, camellia sinensis, thea sinensis, thea bohea, thea assamica, camellia thea, and combinations thereof, together with other compatible agents, flavors, and additives for various specific purposes, as well as the other benefits associated with these plants in conjunction with the effervescent action and properties in the oral cavity.

Oral rinse and mouthwash compositions have been used for many years for the prevention of bad breath and for the elimination of bacteria and other oral micro-organisms that are responsible not only for bad breath, but also tooth decay, caries, plaque, and gum disease such as gingivitis and periodontitis, supragingival plaque accumulation and other decay producing processes. Decay preventing dentifrices have, of course, also been used to and for these ends.

An exciting area of nutritional research is the identification and investigation of compounds, usually derived from or found in plants that can increase our defenses against the agents of disease. Our exposure to dangerous organisms, compounds and toxins has never been greater. In addition to dealing with the oxidative stress of normal metabolism, we must deal with the oxidative pressures of the environment including tobacco smoke, air pollution, and cancer promoting chemicals in our food and water.

Phytochemicals are widely distributed in nature and appear in virtually every plant family. Among the phytochemicals are the polyphenols which include the tannins and condensed tannins such as catechins. One type of catechin is epigallocatechin gallate (EGCG) which is one of the strongest and most studied antioxidants. EGCG is found in white tea, green tea and to a lesser extent in apples. It appears to protect the heart and arteries from oxidative damage.

Catechins are condensed tannins, which are, themselves, one class of a group of protective phytochemicals called polyphenols.

Tea is one of the richest dietary sources of the antioxidant phytonutrients including polyphenols, known to promote sustained vitality and cardiovascular and other health benefits. Jankun, J., Selman, S. H., Sswiercz, R.; Skrzypczak-Jaankun, E.; “Why Drinking the Green Tea Could Prevent Cancer”; Nature, 1997;2387:561.

The following general description of the classification of tea and there relative bioactive compounds is derived from publications of the Linus Pauling Institute for Micronutrient Research for Optimum Health at Oregon State University.

Tea is an infusion of the leaves of the Camellia Sinensis plant. White tea and Green tea are two important forms of the plant Camellia Sinensis. Black tea and Red tea or Oolong tea are other forms. The difference lies in the degree of fermentation after the leaves are picked.

Tea contains a number of bioactive chemicals. Among the chemicals are caffeine, fluoride and the class of compounds known as flavonoids.

While all teas are derived from the leaves of Camellia Sinensis, different processing methods produce different types of tea. Fresh tea leaves are rich in flavonoids known as catechins. Tea leaves also contain polyphenol oxidase enzymes in separate compartments from catechins. When tea leaves are intentionally broken or rolled during processing contact with polyphenol oxidase causes catechins to join together forming dimmers and polymers. These dimmers and polymers are known as theaflavins and thearubigins, respectively. A process of oxidation such as this is known in the tea industry as “fermentation”. Steaming or firing tea leaves inactivates polyphenol oxidase and stops the fermentation process.

There are thousands of tea varieties, but teas are generally divided into three or so groups based on the amount of fermentation undergone during processing.

White teas and Green teas are generally considered to be unfermented teas.

White teas are made from buds and young leaves, which are steamed or fired to inactivate the polyphenol oxidase, and then dried. The White tea thus retains the high concentration of catechins present in the fresh tea leaves.

Green tea is made from more mature tea leaves than White tea and may be withered prior to steaming or firing. For many Green teas, the leaves may be steamed, rolled and dried. This process inactivates the enzymes that cause oxidation.

Oolong teas are considered to be semifermented teas. Tea leaves destined to become Oolong teas are “bruised” to allow the release of some of the polphenol oxidase present in the leaves. These leaves are fermented for less time than Black teas before they are heated and dried. Consequently , the catechin, theaflavin and thearubigin levels are generally between those of unfermented teas such as White tea and Green tea and completely fermented Black teas.

Tea leaves intended to become Black tea are rolled or broken or both to maximize the interaction between catechins and polyphenol oxidase. Since there are completely fermented before drying, most Black teas are rich in theaflavins and thearubigins, but relatively low in catechins.

For Black and Red teas, the leaves are partially dried, then crushed and stored in a temperature and moisture-controlled environment where they oxidize and ferment, longer for the Black tea than for the Red tea.

Polyphenols act as antioxidants, protecting cells and body chemicals against damage caused by free radicals. For example, when low-density lipoprotein (LDL) cholesterol becomes oxidized, it can become glued to the arteries and cause coronary heart disease.

Polyphenols can also block the action of certain enzymes that cancers need for growth.

Teas have, also, been linked to greater bone density and a lower risk of hip fractures.

White tea and Green tea polyphenols are reported to reduce the incidence of, and severity of, rheumatoid arthritis.

Some studies have also suggested that tea consumption may decrease the risk of cataracts.

In addition to the above described benefits, White tea and Green tea is reported to inhibit dental caries and may contain “natural” fluoride or fluoride-like materials preventing the development of caries. In addition, the action of the effervescent bubbles in between the teeth serves to prevent the development of caries.

Before proceeding to the specific examples of the present invention, it is believed that it will be helpful to review the bioactivity of some of the compounds in the various teas referred to above to suggest how these activities may be employed in connection with the present invention.

Flavanols are the most abundant class of flavonoids in tea. Flavanol monomers are also known as catechins.

The principal catechins found in tea are epicatechin (EC), epigallocatechin (EGC), epicatechin gallate (ECG) , and epigallocatechin (EGCG). When catechins are enzymatically oxidized by polyphenol oxidase during fermentation, they form polymers known as theaflavins and thearubigins.

Unfermented teas are rich in catechins, while fermented teas are rich in theaflavins and thearubigins.

Tea is also a good source of another class of flavonoids called flavonois. Flavonols found in tea include kaempferol, quercetin, and myricitin. The flavonol content of tea is less affected by processing and flavonols are present in comparable quantities in fermented and unfermented teas. Unlike flavanols, flavonols are usually present in tea as glycosides which are bount to a sugar molecule.

All teas contain caffeine, unless they are deliberately decaffeinated during processing. The caffeine content of Oolong teas is comparable to Green teas. Buds and young tea leaves have been found to contain higher levels of caffeine than older leaves suggesting that the caffeine content of at least some White teas may be higher that that of Green teas.

A comparison of Green and Black tea with brewed coffee is as follows:

White: Somewhat higher than Green

Green: 40-211 mg/liter

Black: 177-303 mg/liter

Coffee: 306-553 mg/liter.

Tea plants accumulate fluoride in their leaves. In general the oldest tea leaves contain the most fluoride. Most high quality teas are made from the bud or the first four leaves which are the youngest leaves on the plant. The fluoride content of most teas (other than brick tea) is comparable to the levels of fluoride recommended for the prevention of dental caries. The fluoride content of White tea is less that other teas since it is made from the buds and youngest leaves of the tea plant.

A comparison of the fluoride content of several teas is as follows:

White: Lowest

Oolong: 0.6-1.0 mg/liter

Black: 1.0-1.9 mg/liter

Green: 1.2-1.7 mg/liter

Brick: 2.2-7.3 mg/liter.

### EXAMPLES

### Example 1

Typical products may be produced in the following ranges:
4-10% Tea Extract (20-80% Polyphenols)
30-40% Sodium Bicarbonate
25-30% Citric Acid
0.5-2% Sodium Benzoate
15-35% Sorbitol (binder)
2-5% EDTA
0-1% Color
0-2% Flavor

These products may be used as a mouthwash, pre-rinse, breath freshener for humans, pets, and animals and may be drunk, sprayed or irrigated into the oral cavity.

### Example 2

A preferred formulation in accord with the present invention is as follows:
0.150 g Green Tea Extract (50% polyphenols)
0.230 g Sodium Bicarbonate (Baking Soda)
0.190 g Citric Acid
0.0292 g Sorbitol
0.005 g Color (e.g., Mint color)
0.012 Flavor—Spearmint (preferred).

These products may be used as a mouthwash, pre-rinse, breath freshener for humans, pets, and animals and may be drunk, sprayed or irrigated into the oral cavity.

### Example 3

In this example, an alternative, preferred flavor is Mint.

### Example 4

The above formulation has a total weight on the order of 0.600 g from which the percentage compositions may be calculated, as follows:
25% Green Tea Extract (50% polyphenols)
38.3% Sodium Bicarbonate (Baking Soda)
31.6% Citric Acid
4.8% Sorbitol
0.2% Flavor
0.01% Color

### Example 5

Typical products may be produced in the following ranges:
20-30% Tea Extract*(20-80% Polyphenols)
30-40% Sodium Bicarbonate
25-35% Citric Acid
4-25% Sorbitol (binder)
0-1% Color
0-2% Flavor

The tea extract may be a combination of extracts from one or more of the following teas: white, green, oolong, red, and black. The combination may be selected so as to optimize the composition of the end product for specific end users by providing the desired balance and profile of components:
catechins, epicatechin, epigallocatechin, epicatechin gallate, epigallocatechin gallate, theaflavins, thearubigins, flavonols, kaempferol, quercetin, myricitin, glycosides, caffeine, fluoride and the like.

These products may be used as a mouthwash, pre-rinse, breath freshener for humans, pets, and animals and may be drunk, sprayed or irrigated into the oral cavity.

In certain formulations, the presence of a given component may be desired to be minimized (or eliminated—reduced to zero). In other formulations, it may be desired to maximize the specific component. It will be noted in the data set forth above that there is some overlap of the ranges of the data. This fact, of course, means that desired levels of a specific component may be obtained by different formulations of the tea extracts within the scope of the present invention.

The relationship may be generalized as follows:

Σ(Ew+Eg+Eo+Er+Eb)=Σ(C+T+R+A+K+F),

where
Ew=White tea extract
Eg=Green tea extract
Eo=Oolong tea extract
Er=Red tea extract
Eb=Black tea extract, and C=Catechins
T=Theaflavins
R=Thearubigins
A=Flavonols
K=Caffeine
F=Fluoride.

### Example 6

A preferred formulation in accord with the present invention is as follows:
0.150 g Green Tea Extract (50% polyphenols)
0.230 g Sodium Bicarbonate (Baking Soda)
0.190 g Citric Acid
0.0292 g Sorbitol
0.005 g Color (e.g., Mint color)
0.012 Flavor—Spearmint (preferred).

### Example 7

In this example, an alternative, preferred flavor is Spearmint.

### Example 8

The above formulation has a total weight on the order of 600 g from which the percentage compositions may be calculated, as follows:
25% Green Tea Extract (50% polyphenols)
38.3% Sodium Bicarbonate (Baking Soda)
31.6% Citric Acid
4.8% Sorbitol
0.2% Flavor
0.01% Color

These products may be used as a mouthwash, pre-rinse, breath freshener for humans, pets, and animals and may be drunk, sprayed or irrigated into the oral cavity.

### Example 9

Typical products may be produced in the following ranges:
20-30% Tea Extract*(20-80% Polyphenols)
30-40% Sodium Bicarbonate
25-35% Citric Acid
4-25% Sorbitol (binder)
0-1% Color
0-2% Flavor

Tea extract comprising a mixture of white and green tea extracts. These products may be used as a mouthwash, pre-rinse, breath freshener for humans, pets, and animals and may be drunk, sprayed or irrigated into the oral cavity. It should be noted that it is not necessary to add water to start or initiate the reaction.

### Example 10

A preferred formulation in accord with the present invention is as follows:
0.150 g Green Tea Extract (50% polyphenols)
0.230 g Sodium Bicarbonate (Baking Soda)
0.190 g Citric Acid
0.0292 g Sorbitol
0.005 g Color (e.g., Mint color)
0.012 Flavor—Spearmint (preferred).

Tea extract comprising a mixture of white, green and black tea. These products may be used as a mouthwash, pre-rinse, breath freshener for humans, pets, and animals and may be drunk, sprayed or irrigated into the oral cavity.

### Example 11

In this example, an alternative, preferred flavor is Spearmint.

### Example 12

The above formulation has a total weight on the order of 2.605 g from which the percentage compositions may be calculated, as follows:
20-30% Tea Extract * (20-80% Polyphenols)
30-40% Sodium Bicarbonate
25-35% Citric Acid
4-25% Sorbitol (binder)
0-1% Color
0-2% Flavor

The tea extract of Example 10 formulated to provide a 50/50 balance of catechins and the products of fermentation.

The size of the pill, tablet, or capsule may be of any convenient or desired size, typically in the range of from 0.01 g to 5.0 g.

In general, the Tea Extract may range from 0.01% to 40%. The carbonate and acid components may range from 0.01% , when combined in the proper molar ratios, to approximately 60% of the composition. The remaining components may range from 0.0% to approximately 35% where the remaining components and additives in total collectively do not exceed approximately 40% of the total composition.

An effervescent powder, tablet, or capsule of effervescent powder for oral use may be produced with a pharmaceutically acceptable carbon dioxide source selected from the group consisting of a bicarbonate salt, a carbonate salt and mixtures thereof; an organic acid; and polyphenols derived from a plant selected from the group consisting of White tea, Green tea, Oolong tea, Black tea, camellia sinensis, thea sinensis, thea bohea, thea assamica, camellia thea, and combinations thereof, together with other compatible agents, flavors, and additives for various specific purposes,

Sodium bicarbonate, baking soda, has been used for centuries as an aid to clean the human mouth and for deodorizing and freshening the breath. In vitro studies have shown that the bicarbonate ion has mild, but significant antimicrobial properties against anaerobic bacterial species which have been shown to cause periodontal diseases. (Newbrun, E., etal., “Bactericidal Action of Bicarbonate on Selected Periodontal Microoganisms”, Jor. Periodontal 55:11,653-658; 1984). Alkali metal bicarbonates such as sodium bicarbonate are, of course staple items of commerce and can be used together with a peroxide compound, if properly separated, and then combined at the proper time as taught by U.S. Pat. Nos. 4,849,213 and 4,528,180, each of which is herein incorporated by reference.

A glycol ether solvent of liquid ingredients and a sodium perborate carrier for liquid ingredients as taught by U.S. 6,440,906 and 6,451,746, respectively,(each of which is herein incorporated by reference), may also be employed in connection with the present invention.

In addition to citric acid, other acceptable acid sources may be selected from the group consisting of food grade and/or organic acids, such as tartaric acid, fumaric acid, malic acid, phosphoric acid, benzoic acid, and other tricarboxylic acids and partial salts of these acids or mixtures thereof.

Sorbital is a sweetener also known as D-sorbite, D-sorbitol, hexahydric alcohol [C₆H₈(OH)₆]. In addition to, or in lieu of, sorbitol, other sweeteners and/or co-sweeteners may be employed, such as xylital, dextrose, mannitol, fructose and maltose. Artificial sweeteners such as saccharin, aspartame, neohesperidin dihyddrochalcone, malto dextrin and other sweeteners known to the art may, also, be used.

Among the anti-tarter or plaque fighters are compounds such as sodium benzoate, and the like, which may, also, function as preservatives.

White and Green tea are believed to be effective in preventing dental caries. The polyphenols in white and green tea inhibit the growth of Streptococcus mutans which when mixed with food eventually causes plaque. Yamamoto, T. et. al., Chemistry and Applications of Green Tea, CRC Press: New York, 1997. Plaque which is not removed from the teeth mineralizes into calculus or tartar. See, also, Mandel, J. D.: “Chemotherapeutic agents for controlling plaque and gingivitis” J Clin Periodontol. 1988; 15:488-498.

Certain so called essential oils such as thymol, menthol, eucalyptol, anethol, eugenol, caracrol, camphor, anethole, eitronel, a-salpineol, methyl acetate, citronellyl acetate, methyl eugenol, linalool, ethyl linallol, safrola vanillin, spearmint oil, peppermint oil, lemon oil, orange oil, sage oil, rosemary oil, cinnamon oil, pimento oil, laurel oil, cedarleaf oil, clove oil, and methyl salicylate may also be employed for their antiseptic and antiplaque properties, particularly under acidic conditions. Some of these oils may also act as flavoring agents. See U.S. Pat. No. 5,942,211.

The composition can be administered as a tablet, as a powder, and as a capsule or caplet dispersible in human saliva or other aqueous media. The encapsulating coating may include active elements and oils within the gel or in other emulsified and/or encapsulated forms.

The composition of the present invention may also include acceptable carrier materials, orally active metal cations, anti-calculus agents, fluoride ion sources, anti-microbial agents, oral malodor control agents, desensitizing agents, antioxidants, teeth color modifying substances, nutrients, sweeteners, flavor components, coloring agents, polishing agents, bulking agents, fluorine-containing compounds, humectants, thickeners, abrasives, organic surface-active agents and the like.

Fluoride as well as antimicrobial and tarter control agents may also be employed.

Among the antimicrobial actives are phenols, sanguinaria, chlorhexidine, stannous fluoride, sodium fluoride, Cepocol and other antimicrobial agents.

D&C Red 28 (Phloxine B) may also be employed in a preferred formulation. This composition has a CAS Registry Number 018472-87-2 and is known by synonyms: 2′,4′,5′,7′-Tetrabromo-4,5,6,7-tetrachloro-3′,6′-dihydroxyspiro(i-sobenzofuran-1(3H),9′-(9HOxanthen-3-one, disodium salt; Acid red 92; D&CRed no. 28; Eosine I bluish; Fluorescein, 2′,4′,5′,7′-tetrabromo-4,5,6,7,-tetrachloro-, disodium salt; D.I. Acid Red 92. This composition has inherent dental benefits in and of itself in addition to its dye properties. A large number of similar antimicrobial colorants and dyes are know as disclosed in U.S. Pat. No. 6,905,672 which is incorporated herein by reference, as are the teachings of U.S. 4,289,753; 7,083,779; 7,014,862; 6,905,673; 3,629,468; 3,984,342; 4,405,486; 4,409,118; 5,405,836; and 6,984,377.

Well known, essentially anhydrous dentifrice base compounds may be employed. The term “essentially anhydrous” is meant to denote a dentifrice base composition that has a water content sufficiently low so that it will not cause the adsorbed gas to be desorbed from the inorganic oxide material prior to contact with water. The essentially anhydrous dentifrice base medium can include flavor compounds and components, coloring agents, polishing agents, fluorine-containing compounds, humectants, thickeners, abrasives, organic surface-active agents and the like.

One combination of additive elements here presented for purposes of illustration might include 750 mg of Cordyceps Cs (44:1) mushroom extract (Cordyceps sinensis), 200 mg Panax ginsing containing 10% ginsenosides and 200 mg White or Green tea extract (Camellia sinensis) containing 50% polyphenols.

Any suitable flavoring or sweetening agent or mixtures thereof may be employed in formulating a flavor for the compositions of the present invention. Examples of suitable flavoring constituents include the flavoring oils, e.g., oils of spearmint, peppermint, wintergreen, sassafras, cherry, clove, sage, eucalyptus, majoram, cinnamon, lemon, lime, grapefruit, and orange as well as flavoring aldehydes, esters such as methyl salicylate, alcohols, and higher fatty compounds known in the art. Also useful are such chemicals as menthol, carvone and anethole. Of these, the most commonly employed are the oils of peppermint, spearmint, eucalyptus, and anethole, menthol and carvone. In some cases, flavorful solvents, such as chloroform and mock chloroform, may be solidified by being mixed with an acceptable particulate carrier material, such as starch, calcium carbonate, paraffin, vegetable wax, fat, higher fatty acids and other suitable and acceptable carrier substances.

As used herein the term “EDTA” refers ethylenediamineretraacetic acid, a well known organic chelating agent.

Organic surface-active materials may be employed and may be anionic, nonionic, amoholytic, or cationic in nature. It is preferred to employ a surface agent which imparts to the composition detersive and foaming properties. Suitable detergents are water-soluble salts of higher fatty acid monoglyceride monosulfates.

The compositions may also contain common and well known emulsifying and detergent agents.

Components from gas relief and homeopathic compositions may also be employed in accord with the present invention.

In the preparation of powders, it is usually sufficient to admix by milling or other mechanical means the various solid ingredients in appropriate quantities and particle sizes, and thereafter carrying out procedures known in the art for packaging such powdered or tablet compositions. Spray drying and fluid bed mixing may, of course, be employed in the production not only of the powders, per se, but in general in the production of pill, tablets, capsules, caplets, and coated micro-spheres in accordance with the present invention.

The elements of the present invention, except where otherwise noted herein, may be present in the composition in amounts from about 0.01% (weight to weight) to about 5.0% (weight to weight).

A wide variety of natural plant extracts have been shown to exhibit oral odor control benefits, These extracts include extracts obtained from any part of the plant including the leaf, stem, bark, pulp, seed, flesh, juice, root and mixtures thereof. It is preferred that the extract is obtained from the leaf, pulp and seed, more preferably from the leaf or seed. In addition to the teas as indicated above, many different plant, or parts of plants, can be used to provide these extracts including magnolia, gold thread, honeysuckle, grape, bergamot, grapefruit, orange, lemon, tangerine, mandarin, satsuma, clementine, lime and mixtures thereof. Such extracts comprise a wide variety of biologically active materials. These materials include anthocyanins, flavanols, hydrolysable tannins, alkaloids, lipids, carbohydrates, simple sugars, protein and amino acids, alcohols, organic acids and mixtures thereof.

Essential oils are also known to have anti-bacterial properties. These include thymol, geraniol, carvacrol, hinokitiol, eucalyptol, catechol, 4-allyl catechol and mixtures thereof.

Absorbents may also be employed. These are used to absorb, adsorb, bind or otherwise complex the volatile malodor materials. Examples of such agents include talc, mushroom extract, zeolite, cyclodextrin, silica and mixtures thereof.

Pigments such as those from cranberries and/or pomegranate have been found to be effective additions in the compositions of the present invention. The plastid pigments are associated with protoplasmic structure and include water-insoluble chlorophylls, carotene and xanthophylls. The sap soluble pigments include the anthocyanins and anthoxanthins.

In addition to the active agents discussed above the present compositions may contain other anti-plaque/gingivitis agents such as quaternary ammonium compounds, water insoluble agents such as triclosan, stannous salts, and zinc salts. These types of agents are described in U.S. Pat. Nos. 4,894,220; 4,656,031; 5,004,597. All of which are incorporated herein by reference in their entirety.

Abrasive and/or polishing agents may be included in or with the compositions of the present invention. These include silicas, silica gels and precipitates, calcium carbonate, dicalcium orthophosphate dihydrate, calcium pyrophosphate, tricalcium phosphate, calcium polymetaphosphate, insoluble sodium polymetaphosphate, hydrated alumina and resinous abrasive materials. Mixtures of these materials may, also, be used.

Another class of antioxidant polyphenols are found in connection with red and purple pigments called anthocyanins and proanthocyanidins. These are found in berries, cherries, red grapes, plums, pomegranates, red cabbage as well as some beans and grains.

The carotenoids are a further group of antioxidant pigments found in carrots, pumpkins, squash, sweet potatoes, tomatoes, cantaloupes, peaches, mangos, and other yellow and orange fruits and vegetables and most dark, leafy vegetable greens. Of the carotenoids, lutein, zeaxanthin, and lycopene are particularly effective.

The phytoestrogens such as the isoflavones in soy and lignans in flax are also effective and useful.

The polysaccharides found in mushrooms such as shiitake (Lentinula edodes), oyster mushrooms (Pleurotus spp.), enokidake (Flammulina velutipes) and maitake (Grifola frondosa) may be used in accordance with the present invention.

Other phytochemicals such as ellagic acid (raspberries and blueberries), d-limonene (citrus peel), sulforaphane (broccoli), isothiocyanates (in the cabbage family), allicin (garlic) and shogaols (ginger) may also be used in connection with the present invention.

Other additives which may be employed include cretine monohydrate, HMB (b-hydroxy-b-methylbutyrate) and leucine.

Also useful may be DIM (diidolymethane) derived from cruciferous vegetables such as broccoli, cauliflower, cabbage and Brussel sprouts.

MCT (medium chain triglycerides of 5-12 carbon atoms) and evening primrose oils may also be utilized

Vitamins and minerals such as Vitamins A (beta carotene), B, C, D,E, calcium, magnesium, potassium, iron, boron, chromium, manganese, selenium, zinc, and sodium bromide, may be added.

Lipotropics including biotin and choline, inositol, L-carnitine, and betaine may be used.

Coenzyme Q10(CoQ10) desiccated liver, garlic and aspartic acid in various suitable forms may be utilized.

The lignum of the Astronium Urundeura wood from Brazil and the oligomeric proanthocyanidins made by extraction from pine bark may be employed.

A listing of other additives includes: apple polyphenols, rosemary, grape seed extract, epicatechin, eateichin, clovamide, quercetin and their glucosides.

Further additives include potentilla tormentilla, proanthocyaniclins complex, phosphatidyl, serine, pycnogenal, leucocyanidins, pycnogenols, procyanidins, oligomeric, and proanthocyanidins. See U.S. Pat. Nos. 3,436,407; 5,928,646; 5,248,503; 4,698,360.

Other additives include extracts from crab apple, apples, mountain cranberry, avocado, pear stone, cocoa bean, horse chestnut, hawthorn fruit, goat willow, strawberry, raspberry, cola nuts, peanut skins, and pomegranate.

Extracts from ginger, quercetin, rosemary, thyme turmeric, stinging nettle, Noni, Acai, Guarana, Mangosteen, Yerba Mate′, Ardisia Tea and blueberries may be employed in the present invention along with alpha lipoic acid , inositol, glutathione, n-acetyl cysteine,L-taaurine and other amino acids such as found in whey protein.

In general, any flavor enhancers desired may be employed in connection with the present invention. It is also to be noted that depending on the components present the composition may be drunk wy humans as well as pets.

The listings of agents and other components of the composition given in the present specification are not intended to be exhaustive and, therefore, for other materials of these types reference should be made to the standard texts , such as Cosmetics: Science and Technology, by Saccharin, 2^{nd} printing, 1963, published by Interscience Publishers, Inc.

The general benefits of green teas and white teas are becoming increasingly well known and recognized. Recent research suggests hope for polyphenols present in green teas to modulate the insulin-like growth factor-1 (LGF-1) pathway in prostate tumor cells, Mukhtar, Adhami, Siddiqui Ahmad and Gupta, Dec. 1,2007 Cancer Research.

In short, the present invention contemplates the production of an effervescent powder, tablet or capsule of effervescent powder for oral use in which a pharmaceutically acceptable carbon dioxide source selected from the group consisting of a bicarbonate salt, a carbonate salt and mixtures thereof; a food grade or organic acid such as citric acid; and an antiseptically effective amount of phytochemicals, flavonoids, polyphenols and/or antimicrobial agents including those derived from the plant camellia sisensis such as green tea, and more particularly polyphenols derived from a plant selected from the group consisting of White tea, Green tea, Oolong tea, Black tea, camellea sisnsis, thea bohea, thea assamica, camellia thea and combinations thereof.

The food grade or organic acid may be a member selected from the group consisting of citric acid, tartaric acid, fumaric acid, malic acid and partial salts of the acids or mixtures thereof.

The polyphenols may include from 0.01% (weight to weight) to 50% (weight to weight) epigallocatechin gallate (EGCG). EGCG may typically constitute 50% (weight to weight) of a White or Green Tea Extract. The polyphenals may be present in excess of 0.01% (weight to weight) to about 99% (weight to weight), preferably from 0.024% (weight to weight) to 99.98% (weight to weight).

Some authorities hold tea, in general, to be thea sinensis and Green tea, in particular, to be camellia sinensis. (See Sealy, J. R. , A Revision of the Genus Caamella, Section 6, Designated “Thea” 91958). The genus Caamella is said to comprise five species, only three of which are generally connected to the tea plant of commerce.

Many still refer to the tea plant as Thea sinensis even though it is a cultivated member of the camellia family.

It appears that some hold that the correct botanical classification of the tea plant is: Division, angiospermae; Class, dicotyledones; Order, parretales; Family, theaceae; Genus, Thea; Species, sinensis; with over 267,000 separate varieties.

Still others hold the proper classification to be: Division, magnoliophyta; Class, magnoliopsida; Sub-Class, dilleniidae; Order, thealles; Family, theaceae, Sub-Family, camellieae.

Others hold that the proper classification is camellia sisensis (L.), (Kuntze), with thea sinensis. a synonym.

Still others hold that cultivated tea was originally classified as two species, thea sinensis and thea bohea by Linnaeus in 1752. Later it appears the classification was revised with thea sinensis being the designation for the small-leaved Chinese varieties and thea assamica being the designation for the large-leaved Assam plant.

Cultivated tea plants are now often classified as a single genus, camellia, and the family, theaceae (Gutman and Ryu, 1996).

White, Green, Oolong, and Black teas, as well as other varieties which contain polyphenols, flavonols, and methylxanthines may be employed in the present invention. In particular, those containing polyphenols, particularly epigallocatechin gallate (EGCG) and epicatechin as well as catechins such as gallic esters, epicatechin (EC), epicatechin gallate (ECG), also, may be used in the present invention. In light of the above, the applicant acting as his own lexicographer here expresses his desire and intent to define as within the scope of his invention those compositions as set forth above and to be claimed herein as polyphenols, including specifically those derived from a plant selected from the group consisting of White tea, Green tea, Oolong tea, Black tea, camellia sinensis, thea sinensis, thea bohea, thea assamica, camellia thea, and combinations thereof.

Among the specific elements which may be added to the composition of the present invention is a whitener or teeth whitening component. Herein an element of this type is also referred to as a color modifying or teeth color modifying or altering agent.

Some of the components of the various examples of formulations set forth above may in addition serve as whitening agents. For example, baking soda is known to whiten teeth naturally. The effervescent compositions of the present invention may include one or more whitening agents to whiten the color of the teeth. Since many of these whitening agents operate by acting to oxidize or bleach the teeth some care must be directed to avoiding swallowing the composition when these components are a part of the formulation and the agents should be used in relatively minor proportions and with care to avoid burning or otherwise harming the tissues of the oral cavity the gastrointestinal track, or the teeth. These considerations apply not only to human users, but also to pets and other animals.

A natural whitening agent such as baking soda or other substances having the ability to whiten teeth such as those discussed below may be employed. Active compounds suitable for whitening include any material safe for use in the oral cavity which provides whitening, bleaching, or stain removal. Among the actives suitable for whitening are those selected from the group consisting of the peroxides, metal chlorites, perborates, percarbonates, peroxyacids, and combinations thereof. Suitable peroxide compounds include hydrogen peroxide, calcium peroxide, carbamide peroxide, and mixtures thereof. Suitable metal chlorites include calcium chlorite, barium chlorite, magnesium chlorite, lithium chlorite, sodium chlorite and potassium chloride Additional whitening actives may be hypochlorite and chlorine dioxide. Other activies which may be employed include sodium fluoride, sodium monofluorophosphate, pyrophosphate, chlorhexidine, polyphosphate, triclosan, and various enzymes. Examples of appearance and structural changes to be produced include, but are not necessarily limited to: whitening, stain bleaching, stain removal, remineralization, formation of fluorapatite, plaque removal, and tartar removal.

In general, the products of the present invention may be swished vigorously between the teeth for a few seconds up to approximately 30 seconds and then spit out.

As noted above suitable formulations may be drunk by humans. pets and animals. It is also possible to spray or irrigate the teeth and gums with suitable products of the present invention.

The actives may be encapsulated or used in gel forms. They may be used in minor amounts from 0.01% by weight to on the order of 25% of the formulation.

The effervescent composition of the present invention may include alcohol in some minor proportion such as 25% or less or it may be alcohol free. If alcohol is present it should be in an amount not to burn the mouth and the composition should not be swallowed. In general, the product may be swished vigorously between the teeth for a few seconds, up to approximately 30 seconds, and then spit out. A suitably formulated composition, free of alcohol, may be swallowed, if desired. The product suitably formulated may be impregnated in slips to be placed on the tongue or in the mouth or otherwise dropped on the tongue or sprayed in the mouth.

To provide a description of the present invention that is both concise and clear, various examples of ranges have been identified herein with the phrase “including all intermediate ranges and combinations thereof”. Examples of specific values (e.g., %, kDa, ° C., μm, kg/L, Ku) that can be within a cited range by the reference to “including all intermediate ranges and combinations thereof” include 0.000001, 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.1, 1.2, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1050, 1100, 1150, 1200, 1250, 1300, 1350, 1400, 1450, 1500, 1600, 1700, 1800, 1900, 2000, 3000, 4000, 5000, 6000, 7000, 8000, 9000, 10,000, or more.

However, general ranges for each type of unit (e.g., %, kDa, ° C., μm, kg/L, Ku) are contemplated. Examples of values that can be within a cited percentage range, as applicable, include 0.001% to 100%, including all intermediate ranges and combinations thereof. Examples of values that can be within a cited molecular mass range, as applicable, in kilo Daltons (“kDa”), include 0.50 kDa to 110 kDa, including all intermediate ranges and combinations thereof. Examples of values that can be within a cited temperature range, as applicable, in degrees Celsius (“° C.”), that can be within a cited range include of 0° C. to 500° C., including all intermediate ranges and combinations thereof. Examples of values that can be within a thickness range (e.g., coating and/or film thickness upon a surface), as applicable, in micrometers (“μm”), that can be within a cited range include of 1 μm to 2000 μm, including all intermediate ranges and combinations thereof. Examples of values that can be within a cited density range, as applicable, in kilograms per liter (“kg/L”), include 0.50 kg/L to 20 kDa, including all intermediate ranges and combinations thereof. Examples of values that can be within a cited shear rate range, as applicable, in Ku, include 20 Ku to 300 Ku, including all intermediate ranges and combinations thereof.

A matrix of the state of the art and of the level of ordinary skill in the art may be derived by reference to the search terms suggested by the defining elements of the claims.

Other variations and alternative will be apparent to one skilled in the art and are intended to be considered within the scope of the present invention.

### SUMMARY

The present invention comprises an effervescent composition containing an antiseptically effective amount of phytochemicals, flavonoids, polyphenols and/or antimicrobial agents comprising the especially preferred polyphenols derived from a plant selected from the group consisting of White tea, Green tea, Oolong tea, Black tea, camellia sinensis, thea sinensis, thea bohea, thea assamica, camellia thea, and combinations thereof.

An effervescent powder, tablet, or capsule of effervescent powder for oral use is produced containing a pharmaceutically acceptable carbon dioxide source selected from the group consisting of a bicarbonate salt, a carbonate salt, and mixtures thereof; an organic acid and polyphenols derived from a plant selected from the group consisting of White tea, Green tea, Oolong tea, Black tea, camellia sinensis, thea sinensis, thea bohea, thea assamica, camellia thea, and combinations thereof. The product of the present invention may be used by, on or in humans, their pets and/or other animals.

It is noted that the embodiment described herein in detail for exemplary purposes is, of course, subject to many different variations in structure, design, application, and methodology. Because many varying and different embodiments may be made within the scope of the inventive concepts herein taught, and because many modifications may be made in the embodiment herein detailed in accordance with the descriptive requirements of the law, it is to be understood that the details herein are to be interpreted as illustrative and not in a limiting sense. It will be understood in view of the instant disclosure, that numerous variations on the invention are now enabled to those skilled in the art. Many of the variations reside within the scope of the present teachings. It is not intended to cover such alternatives, modifications, and equivalents as may be included within the spirit and scope of the teachings of the present invention. Accordingly, the invention is to be broadly construed and is to be limited only by the spirit and scope of the claims appended hereto.

1. An effervescent product in the form of a powder, tablet, or capsule of effervescent powder for oral use in humans, their pets and other animals comprising:
a) a pharmaceutically acceptable source of carbon dioxide;
b) a food grade or organic acid; and
c) a safe and antiseptically effective amount of flavonoids derived at least in part from White tea.

2. The effervescent product of claim 1 wherein the flavonoids include flavanols.

3. The effervescent product of claim 1 wherein the flavanols include catechins.

4. The effervescent product of claim 3 wherein the product contains other flavonoids derived from a plant selected from the group consisting of White tea, Green tea, Oolong tea, Black tea, camellia sinensis, thea sinensis, thea bohea, thea assamica, camellia thea, derivatives of these materials and mixtures thereof.

5. The effervescent product of claim 3 wherein the product contains flavonoids, phytochemicals, polyphenols antimicrobial agents or chemical compounds and the product further includes one or more of the following: binders, coatings, encapsulating agents, additives, flavors, acceptable carriers, orally active agents, antioxidant, sweeteners, anti-tarter agents, anti-plaque/gingivitis agents, orally active metal cations, anti-calculus agents, fluoride ion sources, anti-microbial agents, oral malodor control agents, desensitizing agents, teeth color modifying agents, nutrients, coloring agents, polishing agents, bulking agents, humectants, thickeners, abrasives, polishing agents, organic surface-active agents, organic chelating agents, emulsifying agents, essential oils, absorbents, pigments, fillers, phytochemicals, vitamins, and minerals.

6. The effervescent product of claim 1 wherein the flavonoids include catechins, theaflavin, and thearubigins.

7. The effervescent product of claim 1 wherein the food grade or organic acid is a member selected from the group consisting of citric acid, tartaric acid, fumaric acid, malic acid, and partial salts of the said acids or mixtures thereof and the carbon dioxide source is at least in part a bicarbonate selected from the group consisting of sodium bicarbonate and potassium bicarbonate or mixtures thereof.

8. The effervescent product of claim 1 wherein the flavonoids are at least in part from an extract derived from the buds and young leaves of White tea.

9. The effervescent product of claim 1 wherein the carbon dioxide source is sodium bicarbonate, the organic acid is citric acid and the product contains an extract derived from White tea, Green tea or both.

10. The effervescent product of claim 2 wherein the carbon dioxide source is sodium bicarbonate, the organic acid is citric acid and the product contains White tea.

11. The effervescent product of claim 2 wherein the said polyphenols include from 0.01% to 50% epigallocatechin gallate.

12. The effervescent product of claim 2 wherein the polyphenols are derived at least 50% from a White tea extract.

13. The effervescent product of claim 2 wherein the flavonoids are present in excess of 0.01%.

14. The effervescent product of claim 2 wherein the flavonoids are present from 0.01% to 99.9%.

15. An effervescent product in the form of a powder, tablet, capsule, film, strip or coating including an effervescent powder for oral use in humans, their pets and animals comprising:
a) a pharmaceutically acceptable source of carbon dioxide selected from the group consisting of a bicarbonate salt, a carbonate salt, and mixtures thereof;
b) a food grade or organic acid selected from the group consisting of citric acid, tartaric acid, fumaric acid, malic acid and partial salts of the acids or mixtures thereof; and
c) a safe and antiseptically effective amount of flavonoids, derived from a plant selected from the group consisting of White tea, Green tea, Oolong tea, Black tea, camellia sinensis, thea sinensis, thea bohea, thea assamica, camellia thea, derivatives of these materials and mixtures thereof wherein the flavonoids, phytochemicals, polyphenols antimicrobial agents or chemical compounds from these plants are present in an amount from 0.024% to 99.98% including flavanols, catechins, theaflavins, thearubigins, epicatechin, epigallocatechin, epicatechin gallate, epigallocatechin gallate, flavonols, kaempferol, quercetin, myricitin, or glycosides.

16. The effervescent product of claim 15 wherein The effervescent product of claim 1 wherein the product includes one or more of the following: binders, coatings, encapsulating agents, additives, flavors, acceptable carriers, orally active agents, antioxidant, sweeteners, anti-tarter agents, anti-plaque/gingivitis agents, orally active metal cations, anti-calculus agents, fluoride ion sources, anti-microbial agents, oral malodor control agents, desensitizing agents, teeth color modifying agents, nutrients, coloring agents, polishing agents, bulking agents, humectants, thickeners, abrasives, polishing agents, organic surface-active agents, organic chelating agents, emulsifying agents, essential oils, absorbents, pigments, fillers, phytochemicals, vitamins, and minerals.

17. The method of delivering an antiseptically effective amount of flavonoids for oral use comprising presenting an effervescent product in the form of a powder, tablet, capsule, film, strip, or coating including an effervescent powder for oral use in humans, their pet and animals, comprising:
a) providing a pharmaceutically acceptable source of carbon dioxide;
b) providing a food grade or organic acid subject to activation by an aqueous medium; and
c) a safe and antiseptically effective amount of flavonoids, derived from a plant selected from the group consisting of White tea, Green tea, Oolong tea, Black tea, camellia sinensis, thea sinensis, thea bohea, thea assamica, camellia thea, derivatives of these materials and mixtures thereof wherein a safe and effective amount of the flavonoids, phytochemicals, polyphenols antimicrobial agents or chemical compounds from these plants are present in an amount from 0.024% to 99.98% including flavanols, catechins, theaflavins, thearubigins, epicatechin, epigallocatechin, epicatechin gallate, epigallocatechin gallate, flavonols, kaempferol, quercetin, myricitin, glycosides, caffeine or fluoride to provide a desired profile of at least the catechins and the products of fermentation.

18. The method of claim 17 wherein the product includes one or more of the following: binders, coatings, encapsulating agents, additives, flavors, acceptable carriers, orally active agents, antioxidant, sweeteners, anti-tarter agents, anti-plaque/gingivitis agents, orally active metal cations, anti-calculus agents, fluoride ion sources, anti-microbial agents, oral malodor control agents, desensitizing agents, teeth color modifying agents, nutrients, coloring agents, polishing agents, bulking agents, humectants, thickeners, abrasives, polishing agents, organic surface-active agents, organic chelating agents, emulsifying agents, essential oils, absorbents, pigments, fillers, phytochemicals, vitamins, and minerals.

19. The method of claim 18 wherein additional phytochemicals serve as oral malodor control agents.

20. The method of claim 18 wherein at least some of the active agents are encapsulated.
